SEQUENCE LISTING (19 sequences)

SEQ ID NO: 1 - protein, 30 aa, Artificial Sequence
  The sequence is developed from MPT63 of M. tuberculosis
GQVWEATATVNAIRGSVTPAVSQFNARTAD

SEQ ID NO: 2 - protein, 25 aa, Artificial Sequence
  The sequence is developed from MPT63 of M. tuberculosis
ATATVNAIRGSVTPAVSQFNARTAD

SEQ ID NO: 3 - protein, 25 aa, Artificial Sequence
  The sequence is developed from MPT63 of M. tuberculosis
GQVWEATATVNAIRGSVTPAVSQFN

SEQ ID NO: 4 - protein, 20 aa, Artificial Sequence
  The sequence is developed from MPT63 of M. tuberculosis
ATATVNAIRGSVTPAVSQFN

SEQ ID NO: 5 - protein, 15 aa, Artificial Sequence
  The sequence is developed from MPT63 of M. tuberculosis
NAIRGSVTPAVSQFN

SEQ ID NO: 6 - protein, 15 aa, Artificial Sequence
  The sequence is developed from MPT63 of M. tuberculosis
ATATVNAIRGSVTPA

SEQ ID NO: 7 - protein, 10 aa, Artificial Sequence
  The sequence is developed from MPT63 of M. tuberculosis
NAIRGSVTPA

SEQ ID NO: 8 - protein, 19 aa, Artificial Sequence
  The sequence is developed from MPT63 of M. tuberculosis
GNVIRGSVAPAVSQFNGPL

SEQ ID NO: 9 - protein, 19 aa, Artificial Sequence
  The sequence is developed from MPT63 of M. tuberculosis
PQNAIRGAVTPAVSQFNVA

SEQ ID NO: 10 - protein, 19 aa, Artificial Sequence
  The sequence is developed from MPT63 of M. tuberculosis
PQNAIRWSVTPAVSQFNLH

SEQ ID NO: 11 - protein, 20 aa, Artificial Sequence
  The sequence is developed from MPT63 of M. tuberculosis
DFNAIVGSVTPAVSQFNGAL

SEQ ID NO: 12 - protein, 20 aa, Artificial Sequence
  The sequence is developed from MPT63 of M. tuberculosis
LKNPIRGSQTPAVSQFNHIG

SEQ ID NO: 13 - protein, 19 aa, Artificial Sequence
  The sequence is developed from MPT63 of M. tuberculosis
GANAIRGSHDPAVSQFNGL

SEQ ID NO: 14 - protein, 19 aa, Artificial Sequence
  The sequence is developed from MPT63 of M. tuberculosis
GANAIRCSVFPAVSQFNIG

SEQ ID NO: 15 - protein, 40 aa, Artificial Sequence
  The sequence is developed from MPT63 of M. tuberculosis
GYPVAGQVWEATATVNAIRGSVTPAVSQFNARTADGINYR

SEQ ID NO: 16 - protein, 20 aa, Artificial Sequence
  The sequence is developed from MPT63 of M. tuberculosis
PQGEQSTGKIYFDVTGPSPT

SEQ ID NO: 17 - DNA, 20 bp, Artificial Sequence
  PCR Primer
tcggcagaacaccattgtgg

SEQ ID NO: 18 - DNA, 21 bp, Artificial Sequence
  PCR Primer
gttcagggtgatgagcgtgac

SEQ ID NO: 19 - protein, 519 aa, Homo sapiens
MMSEHDLADVVQIAVEDLSPDHPVVLENHVVTDEDEPALKRQRLEINCQDPSIKTICLRLDSIEAKLQALEATCKSLEEKLDLVTNKQHSPIQVPMVAGSPLGATQTCNKVRCVVPQTTVILNNDRQNAIVAKMEDPLSNRAPDSLENVISNAVPGRRQNTIVVKVPGQEDSHHEDGESGSEASDSVSSCGQAGSQSIGSNVTLITLNSEEDYPNGTWLGDENNPEMRVRCAIIPSDMLHISTNCRTAEKMALTLLDYLFHREVQAVSNLSGQGKHGKKQLDPLTIYGIRCHLFYKFGITESDWYRIKQSIDSKCRTAWRRKQRGQSLAVKSFSRRTPNSSSYCPSEPMMSTPPPASELPQPQPQPQALHYALANAQQVQIHQIGEDGQVQVGHLHIAQVPQGEQVQITQDSEGNLQIHHVGQDGQLLEATRIPCLLAPSVFKASSGQVLQGAQLIAVASSDPAAAGVDGSPLQGSDIQVQYVQLAPVSDHTAGAQTAEALQPTLQPEMQLEHGAIQIQ

(19) United States
(12) Patent Application Publication
(10) Pub. No.: US 2019/0382454 A1
(43) Pub. Date: Dec. 19, 2019

(54) A MODIFIED PEPTIDE AS AN ANTICANCER AGENT

(71) Applicant: Amrita Therapeutics Limited, Ahmedabad (IN)

(72) Inventors: Samit CHATTOPADHYAY, Pune (IN); Nandaraj TAYE, Pune (IN); Prabhakar SALUNKHE, Jalgaon (IN)

(21) Appl. No.: 15/555,819

(22) PCT Filed: Mar. 7, 2016
(86) PCT No.: PCT/IN16/50081

§ 371 Date: Sep. 5, 2017

(30) Foreign Application Priority Data
Mar. 5, 2015 (IN) 740/MUM/2015

(51) Int. Cl. C07K 14/35

(52) U.S. Cl. CPC C07K 14/35; A61K 38/00

(57) ABSTRACT

A modified peptide AT-01 has been synthesized from the microbiomic secretory protein MPT63 of Mycobacterium tuberculosis. This peptide (about 30 amino acids) contains a portion of the immunogenic region of MPT63 (131 amino acids). It has been found to stabilize SMAR1 (Scaffold Matrix Attachment Region Binding Protein 1), a tumor suppressor protein which is well characterized as anti-cancer. AT-01 was modified further to generate 6 new, independent peptides. Among the peptides AT-01 C and AT-01 D were found to be more effective than the other peptides. AT-01 D also attenuated cell migration of MDA-MB231 cells which supports the anti-metastatic activity. Inhibition of the cell growth from the Colony Formation Assays too supported its anticancer activity. It does not cause toxicity to the cells at high doses which demonstrates great potential value as a safe and effective cancer therapy.

### FIELD OF THE INVENTION

The present invention particularly relates to a novel peptide synthesized from the secretory protein MPT63 of the microbiome bacterium Mycobacterium tuberculosis. Particularly the invention provides a peptide having antitumor anticancer activity. More particularly, the peptide of the present invention stabilizes Scaffold Matrix Attachment Region Binding Protein 1 (SMAR1), which further suppresses tumor. The peptide AT01 containing about 30 amino acids is further modified to develop a potential anticancer agent with enhanced anticancer activity.

### BACKGROUND OF THE INVENTION

Cancer is emerging as a leading global public health threat for developing and developed countries alike. The American Cancer Society projects diagnosis of nearly 1.66 million new cancer cases diagnosed in 2015, and an estimated 589,430 cancer deaths in the United States alone. Cancer progression is characterized by uncontrolled cell growth and proliferation of mutated cells, where fast-growing cancers may result in death of the patient if left untreated. To date, cancer interventions have relied primarily on radiotherapy, immunotherapy and chemotherapy approaches. By and large, these cancer therapies available in the market today are both costly and/or cause severe side effects. In addition, these therapies may result in development of resistance of the cancer or may sow the seeds of eventual recurrence of cancer that will be resistant to continued therapy. Hence, there is an urgent need for alternative therapeutic approaches that are both safer and cost-effective for patients, where Amrita Therapeutics' modified peptides may provide one solution for this major unmet medical need.

Despite tremendous efforts in molecular, biochemical and cell biological research towards understanding the intra- and extra-cellular mechanisms involved in the transformation of a normal cell into a cancerous one, yet the world awaits a truly sustainable, cost-effective, safe and effective therapy for cancer. A major limitation for many cancer therapeutics is the problem of delivering pharmacologically relevant compounds, peptidyl mimetic, antisense oligonucleotides, and proteins into cells (Egleton and Davis, 1997). Peptide-based drugs have limitations in the form of the poor permeability and selectivity of the cell membrane. These problems are now circumvented by attaching protein translocation domains (PTDs) to the peptides that can cross the biological membranes efficiently without any dependence on transporters or specific receptors and mediate the intracellular delivery of a range of biological cargos.

Neoplastic progression requires multiple genetic mutations, where inactivation of p53 is a commonly associated with approximately 60% of all human cancers (Levin, 1997; Michael and Oren, 2002). The tumor suppressor protein p53 is a short lived, latent transcription factor that is activated and stabilized in response to a wide range of cellular stresses, including DNA damage and activated oncogenes. p53 participates in the regulation of several processes, which might inhibit tumor growth, including differentiation, senescence and angiogenesis (Vogelstein et al., 2000; Oren, 2003). However, the central function of p53 appears to be the ability to induce both cell cycle arrest and/or apoptosis in stressed cells, partly by activating expression of p53 responsive target genes that mediate these responses. The precise mechanism of p53 activation by cellular stress is of intense interest and may involve both increases in p53 protein level and in the specific activity of p53 by covalent modifications, where highly conserved residues in its N- and C-terminal domains are targets for potential post-translational modifications via phosphorylation, ubiquitination or acetylation (Giaccia and Kastan, 1998; Michael and Oren 2003).

The present investigations were aimed at developing one or more peptides that upregulate p53 by stabilizing tumor suppressor protein SMAR1 both in vitro and in vivo. SMAR1 is a 68 KDa protein reported to be a potent tumor suppressor that prevents tumor growth and progressions, playing the role of a master regulatory in the cell. It interacts and stabilizes the p53 serine-15 that precludes tumor growth by regulating the cancer cell growth. SMAR1 is known to suppress Cyclin D1 gene and prevents the cell progression by arresting the cell at G1 phase. Cyclin D1 gene that is required in the transition of G1 to S phase is abnormally active during cancers. Cyclin D1 also has been found to be over-expressed in breast carcinoma. This protein has been shown to interact with tumor suppressor protein Rb and the expression of this gene is regulated positively by Rb. Mutations, amplification and overexpression of this gene, which alters cell cycle progression, is observed frequently in a variety of tumors and may contribute to tumorigenesis. This highlights the immense importance of SMAR1 in regulation of tumorigenesis. In higher grades of breast cancers, SMAR1 is reported to be down-regulated in proportion to the stage of the breast cancer. This research indicates that SMAR1 is degraded or lost entirely as cancers develop and needs to be restored to control the abnormal cancer cell growth. Our research demonstrates for the first time the effectiveness of AT-01 to actually bind to SMAR1 and to prevent the degradation of SMAR1, which in turn maintains the expression level required to check abnormal cell growth and progressions. The peptide interventions of AT-01C and AT-01D in particular demonstrate enhanced efficacy needed to enable SMAR1 expression to be maintained inside the cell so as to carry out its role as master cell regulator to down-regulate oncogenes, ie to continue SMAR1's traditional anti-cancerous activities. Thus the AT-01C and AT-01D peptides may serve as useful agents as oncology peptide therapeutics in cancers especially by stabilizing tumor suppressor proteins like SMAR1.

### OBJECT OF THE INVENTION

(i) The main objective is to provide a further novel peptide that can act as an anti-cancer agent eliminating/minimizing the limitations of prior art.
(ii) The second objective is to provide a peptide synthesized from the secretary protein MPT63 of Mycobacterium tuberculosis.
(iii) The third objective is to provide the peptide that stabilizes the master cell regulatory protein known as Scaffold Matrix Attachment Region Binding Protein 1 (SMAR1), which further suppresses oncogenes and reactivates p53. Further, the peptide will maintain the expression level of SMAR1 in the cell that will execute anti-cancerous activity.
(iv) The fourth objective is to provide safe, user-friendly therapy via modified peptide AT-01 containing about 30 amino acids or derivative thereof with enhanced anticancer activity.
(v) The fifth objective is to provide modified peptide AT-01 or derivative thereof useful as a potential therapeutic agent or to develop peptidyl drug to arrest cancer.
(vi) The sixth objective is to develop modified AT-01 or derivative thereof useful as a potential imaging or diagnostic agent for early detection of cancer.
(vii) The seventh objective is to provide compositions, pharmaceutical compositions containing AT-01, modified AT-01 peptide or derivative thereof and their applications.
(viii) The eighth objective is to provide a diagnostic, prophylactic or treatment method of a subject suffering from cancer.

### DESCRIPTION OF THE DRAWINGS

FIG. 1: depicts the structure of peptide derivatives (AT-01A to AT-01F)

FIG. 2a: Depicts in vitro treatment of HCT116 cells with AT-01, AT-01A to AT-01F, with heightened effectiveness of AT-01C and AT-01D in stabilizing SMAR1

FIG. 2b to f: Illustrates heightened effectiveness of peptide derivative AT-01D in stabilizing SMAR1, as demonstrated across a variety of cancer cell lines

FIGS. 3a to 3k: Isothermal Titration Colorimetry (ITC) experiments demonstrating the physical interaction between SMAR1 protein of either full length, Protein binding domain (PBD) or DNA binding domain (DBD) with the AT-01 and derived peptides.

FIG. 4: Depicts potential binding site of AT-01C and AT-01D peptides with SMAR1 as predicted by computer visualization software

FIG. 5: Shows mitigation of anticipated cell migration of Human Breast Carcinoma Cell Line (MDA-MB231) in presence of AT-01C and AT-01D peptides utilizing colony formation assay

FIG. 6: Illustrates effect of AT-01C during in vivo pre-clinical trials demonstrating reduction in tumor volume by one-half to two-thirds during the period of treatment of HT29 and HCT116 colon cancer cells respectively.

### SUMMARY OF THE INVENTION

Accordingly the present invention provides a modified peptide as an anticancer agent with increased anticancer activity consisting of amino acid sequence of SEQ ID No 1 and its variants/derivatives and pharmaceutical composition thereof.

According to one of the embodiments, the variant comprising peptides designated as AT01A, AT01B, AT01C, AT01D, AT01E, and AT01F derived from AT01 have the SEQ ID NO. 2, 3, 4, 5, 6 and 7 respectively.

According to second embodiment, the variant comprising peptides designated as AT01C have the SEQ ID NO. 4.

According to third embodiment, the variant comprising peptides designated as AT01D have the SEQ ID NO. 5.

According to fourth embodiment, the peptide, an anticancer agent may be synthesized from a secreted protein MPT63 of M. tuberculosis.

According to fifth embodiment the invention provides a pharmaceutical composition comprising AT01 or variants thereof d.

According to sixth embodiment the invention provides a pharmaceutical composition further comprising pharmaceutically acceptable carriers and/or adjuvants wherein the peptide may range from 0.1 to 250 mg/ml.

According to seventh embodiment the invention provides a pharmaceutical composition further comprising other conjugating agents responsible for targeted delivery and/or diagnostic applications.

According to eighth embodiment the invention provides a pharmaceutical composition that may be useful to stabilize Scaffold Matrix Attachment Region Binding Protein 1 (SMAR1), which further suppresses tumor through cell cycle arrest by suppressing Cyclin D1.

According to ninth embodiment the invention provides a method of diagnosing preventing and treating a subject suffering from cancer by administrating the peptide AT01 or their variants optionally with other conjugates or a pharmaceutical composition thereof.

### DETAILED DESCRIPTION

Various secretory proteins are secreted by M. tuberculosis that allow the gain of entry and favour infections into the host system. Goulding et al (2002) have identified 3,924 secretory proteins in M. tuberculosis using bioinformatics approach. MPT-63 a secretory protein from M. tuberculosis exhibits anti-cancer activities. It is a 17 KDa protein with unknown functions apart from its immunogenic activities. But its 1.5 angstrom crystal structure has been well described in the Protein Data Bank.

A 30 amino acid peptide designated as AT01 is synthesized from MPT-63 that shows anti-cancer property. This peptide was further modified from either side to pin-point the exact sequence required for its anti-cancer activities. Thus, six peptides were generated from the parent 30 amino acid peptide i,e AT-01. These peptides were designated as AT-01 (30 amino acids), AT-01A (25 amino acids), AT-01B (25 amino acids), AT-01C (20 amino acids), AT-01D (20 amino acids), AT-01E (15 amino acids) and AT-01F (10 amino acids). Additional peptides named DM01, DM02, DM03, DM04, DM05, DM06, DM07, EXT1, AND EXT2 having SEQ ID numbers 8, 9, 10, 11, 12, 13, 14, 15, and 16 respectively were designed by the selective replacement or addition of amino acids in AT-01 as shown in Table 1.

TABLE 1

| | | SVM | | Steric | | | |
|---|---|---|---|---|---|---|---|
| Name | Sequence | score | Prediction | hindrance | Hydrophobicity | Charge | pI |
| AT-01 | GQVWEATATVNAIRGSVTPAVSQ | 0.63 | Anticp | 0.61 | −0.09 | 0.00 | 6.42 |
| | FNARTAD | | | | | | |
| AT-01A | ATATVNAIRGSVTPAVSQFNART | 0.72 | Anticp | 0.61 | 0.05 | 1.00 | 9.95 |
| | AD | | | | | | |
| AT-01B | GQVESATATVNAIRGSVTPAVSQ | 0.55 | Anticp | 0.62 | 0.09 | 0.00 | 6.36 |
| | FN | | | | | | |
| AT-01C | ATATVNAIRGSVTPAVSQFN | 0.70 | Anticp | 0.61 | 0.32 | 1.00 | 10.11 |
| AT-01D | NAIRGSVTPAVSQFN | 0.69 | Anticp | 0.62 | 0.00 | 1.00 | 10.11 |
| AT-01E | ATATVNAIRGSVTPA | 0.68 | Anticp | 0.59 | 0.48 | 1.00 | 10.11 |
| AT-01F | NAIRGSVTPA | 0.67 | Anticp | 0.60 | 0.08 | 1.00 | 10.11 |
| DMO1 | GNVIRGSVAPAVSQFNGPL | 0.97 | Anticp | 0.59 | 0.00 | 1.00 | 10.11 |
| DMO2 | PQNAIRGAVTPAVSQFNVA | 0.63 | Anticp | 0.58 | −0.04 | 1.00 | 10.11 |
| DMO3 | PQNAIRWSVTPAVSQFNLH | 0.89 | Anticp | 0.57 | −0.10 | 1.50 | 10.11 |
| DMO4 | DFNAIVGSVTPAVSQFNGAL | 0.80 | Anticp | 0.63 | 0.09 | −1.00 | 3.80 |
| DMO5 | LKNPIRGSQTPAVSQFNHIG | 0.67 | Anticp | 0.56 | −0.15 | 2.50 | 11.10 |
| DMO6 | GANAIRGSHDPAVSQFNGL | 0.61 | Anticp | 0.59 | −0.09 | 0.50 | 7.10 |
| DMO7 | GANAIRCSVFPAVSQFNIG | 0.53 | Anticp | 0.63 | 0.03 | 1.00 | 8.60 |
| EXT1 | GYPVAGQVWEATATVNAIRGSVT | 0.71 | Anticp | 0.62 | −0.09 | 1.00 | 8.39 |
| | PAVSQFNARTADGINYR | | | | | | |
| EXT2 | PQGEQSTGKIYFDVTGPSPT | 0.85 | Anticp | 0.60 | −0.14 | −1.00 | 4.38 |

In order to understand and confirm the role of these peptides in the cancer progression pathway, Scaffold Matrix Attachment Region Binding Protein 1 (SMAR1) induction was tested. SMAR1 is recognized as a master cell regulator, and one of the nuclear matrix associated proteins whose expression is drastically reduced in higher grades of many cancers including breast cancer. SMAR1 gene is located on human chromosome 16q24.3 locus, the loss of heterozygosity (LOH) of which has been reported in several types of cancers (Malonia et al., 2011). SMAR1 is also reported to be a p53-interacting protein that is involved in delaying tumor progression in vivo as well as in regulating the cell cycle. It has been demonstrated that SMAR1 physically interacts and co-localizes with p53 (Jalota et al., 2005). These peptides bind in the pocket of SMAR1 tightly and avoid its ubiquitination. In order to identify the smallest and best functional region of this 30 amino acid peptide for SMAR1 stabilization, additional peptides were designed by deleting 5 amino acids from each side of AT01. In this way, two peptides of 25 amino acids (AT-01A & AT-01B), one peptide of 20 amino acids (AT-01C), two peptides of 15 amino acids (AT-01D & AT-01E) and one peptide of 10 amino acids (AT-01F) were prepared and screened for the SMAR1 induction assays. The smallest peptides demonstrating superior induction of SMAR1 were AT-01C and AT-01D. We believe that many of these peptides will show induction of SMAR1.

Although MPT63 secretory protein is harmful and infectious in nature, it nonetheless plays an important biological role especially with regard to genetic modifications. In this context, the inventors have for the first time developed empirical evidence proving the anti-cancer properties of these peptides for the first time, and have demonstrated that these peptides in fact stabilize tumor suppressing protein SMAR1 by preventing degradation of the SMAR1 protein that in turn down-regulates oncogenes and causing tumor reduction.

These peptides can be used alone or in conjunction with other compounds. As one example, they can be conjugated to drugs in targeted drug delivery systems. As yet another example, they can be conjugated to probes for diagnostic or research applications.

The peptide(s) of the invention may also be administered in combination with agents, such as methotrexate, 6-MP, azathioprine sulphasalazine, mesalazine, olsalazine chloroquinine/hydroxychloroquine, penicillamine, aurothiomalate (intramuscular and oral), azathioprine, colchicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors, TNFα converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™) and p55TNFRIgG (lenercept), sIL-1RI, sIL-1RII, and sIL-6R), anti-inflammatory cytokines (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, human recombinant, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl, misoprostol, naproxen sodium, omeprazole, cyclophosphamide, rituximab, IL-1 TRAP, MRA, CTLA4-IG, IL-18 BP, anti-IL-18, anti-IL15, BIRB-796, SCIO-469, VX-702, AMG-548, VX-740, Roflumilast, IC-485, CDC-801, and Mesopram.

Non-limiting examples of therapeutic agents for cancers with which a peptide of the invention can be co-administered or used in combination include the following: budenoside; epidermal growth factor; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1.beta. monoclonal antibodies; anti-IL-6 monoclonal antibodies; growth factors; elastase inhibitors; pyridinyl-imidazole compounds; and antibodies to or antagonists of other human cytokines or growth factors, for example, TNF, LT, IL-1, IL-2, IL-6, IL-7, IL-8, IL-15, IL-16, IL-17, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90, or their ligands.

The present invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The present invention may additionally comprise administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, a composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml, or at least 100 mg/ml. The liquid form should be stored at between 2° C. and 8° C. in its original container.

The peptide of the invention can be incorporated into a pharmaceutical composition suitable for parenteral administration. Preferably, the peptide will be prepared as an injectable solution containing 0.1-250 mg/ml. The injectable solution can be composed of either a liquid or lyophilized dosage form in a flint or amber vial, ampoule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In an embodiment, a peptide described herein is administered by intravenous infusion or injection. In another embodiment, the peptide is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The peptide of the present invention can be administered by a variety of methods known in the art, although for many therapeutic applications, the preferred route/mode of administration is subcutaneous injection, intravenous injection, or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. In certain embodiments, a binding protein of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, a binding protein of the invention is co-formulated with and/or co-administered with one or more additional therapeutic agents that are useful for treating disorders in which peptide activity is detrimental. Furthermore, one or more binding proteins of the invention may be used in combination with two or more of the foregoing therapeutic agents. Such combination therapies may advantageously utilize lower dosages of the administered therapeutic agents, thus avoiding possible toxicities or complications associated with the various monotherapies.

### Materials and Methods

### Peptide Synthesis

All the peptides used in the study were synthesized chemically by United Biosystems Inc (Herndon, USA). The peptides were synthesized by solid phase chemistry and purified to more than 95%. The purity was checked by HPLC and Mass Spectrometry. The peptides were received in the lyophilized form and dissolved in the buffer to get the required stock solution.

### Docking and Bio-Informatics

Docking analysis has been done using Autodock software in order to find out the precise interacting residues of SMAR1 and the peptides. The peptide structures were predicted by software and the PDB files generated were used for the subsequent docking analysis. Analysis of the interacting amino acids were done using PDB sum to find out the bond length and the binding residues. Physiochemical properties of the peptides were obtained using various bio-informatics software.

### Cell Culture and Peptide Treatment

SW620, MCF7, HCT116p53^{+/+} and HCT116 p53^{-/-} cells were maintained in DMEM medium (GIBCO) and MDA-MB231 cells in L-15 DMEM medium. NCI-H522 was cultured in RPMI 1640 medium (GIBCO). All the cells were supplemented with 10% Fetal Bovine Serum (FBS) and 100 Units of Penicillin Streptomycin antibiotics. Culture was maintained in a 37° C. incubator supplied with 5% carbon-dioxide gas and maintained at 97% relative humidity. MDA-MB231 cells were maintained in carbon-dioxide free incubator keeping the other conditions same. Cells were seeded and next day treated with peptide for 48 hours. After 24 hours fresh medium was added and peptide treatment was given again for another 24 hours.

### SDS PAGE and Western Blotting

A 10% SDS PAGE was used to resolve the proteins and transferred into a PVDF membrane by western blotting technique. Blot was overnight blocked with 5% BSA at 4° C. Next day blot was washed with 1×TBST and primary antibody was added for 2 hours at RT. It was followed by washing and addition of secondary antibody for 1 hour at RT. Blot was again washed and developed by auto-radiography in an X-Ray film (Kodak). Antibodies used were SMAR1 (Bethyl), β-Actin (Sigma).

### PCR

HCT116p53^{+/+} (3×10^{\hat{ }5}) cells were cultured in 35 mm petri dishes and treated with different concentrations of the peptide for 48 hours. mRNA was isolated using Trizol (Invitrogen) and estimated in Nanodrop. cDNA was prepared from 2 μg of mRNA and PCR was set up using 2 μl of the cDNA. SMAR1 primers used for the PCR are Forward Primer TCGGCAGAACACCATTGTGG and Reverse primer is GTTCAGGGTGATGAGCGTGAC. GAPDH was used as loading control. PCR conditions were maintained at 95° C. for 1 min, 62° C. for 1 min and 72° C. for 35 cycles. Amplicons were run in 1% Agarose gel and stained with Ethidium Bromide reagent. Gel was exposed with UV light transilluminator (Bio-Rad) and documented.

### Peptide Interaction Assay by ITC

SMAR1 full length and the truncated (Protein Binding and DNA Binding Domain) SMAR1 were expressed, purified and used for interaction studies with the peptides by Isothermal Titration calorimetry (ITC) Assay. All the peptides were titrated with the full length and truncated SMAR1 proteins, followed by measuring the thermal change by ITC. The binding constants were calculated from the data obtained from ITC. Set up was done at 25° C. using ITC instrument unless mentioned.

### Wound Healing Assay

MDA-MB231 breast cancer cells (1×10^{\hat{ }6}) were seeded in a 6 well plate and maintained in L15 DMEM medium supplemented with 10% FBS and 100 Units of Penicillin Streptomycin antibiotics for 24 hours. Cells were treated for 24 hours with AT-01C and AT-01D peptides of 10 μg each. Post 24 hours wound was created and washed with medium to remove the cells detached during wound creation. Fresh peptides were added plate was mounted at the microscope chamber maintaining the temperature of 37° C. Images were acquired in NIKON Confocal Microscope for 5 minutes interval. Time lapse was set for 16 hours and images were compiled to make the video movie of the cell migration.

### Colony Formation Assay

200 HCT116 cells seeded in 35 mm petri dishes and cultured for 24 hours. 10 μg of AT-01D added fresh every two days up to day 12^{th}. Cell were washed with 1× Phosphate Buffer Saline and then fixed using 3% Para-formaldehyde for 10 minutes. Cells were washed and stained with Crystal violet for 30 minutes. After washing with PBS plates were kept inverted and air dried. Colonies were counted using inverted bright field microscope (Nikon). A group of cells were identified to be a colony that consists of more than 50 cells.

### In Vivo Experiments

SCID mice were injected subcutaneously with either 1 million HCT116 cells (carcinoma) or HT29 cells (adenocarcinoma) both of which are colon cancer cells. After 2 weeks tumors developed could be seen and the process of injecting AT-01C peptide was initiated. For HCT116 xenograft mice model AT-01C peptide of 25 mg/kg body weight of mice was injected intra-peritoneally. In case of HT29 xenograft mice model AT-01C peptide of 50 mg/kg body weight of mice was injected intra-peritoneally. Stock concentration of the AT-01C was 10 mg/ml. Both the mice group were injected with AT-01C peptide for 21 days daily just 5 mm away from the tumors. The daily injection was performed by injecting AT-01C surrounding the tumors being developed. After 21 days of injection tumors excised and their weight and volume was measured.

AT-01C Regressed Tumor Formation in SCID Mice:

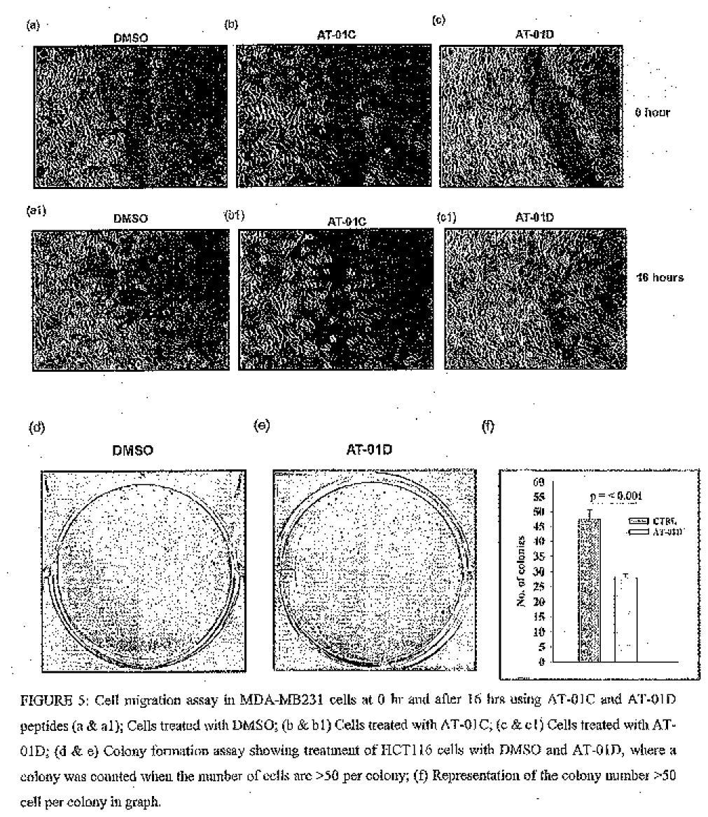

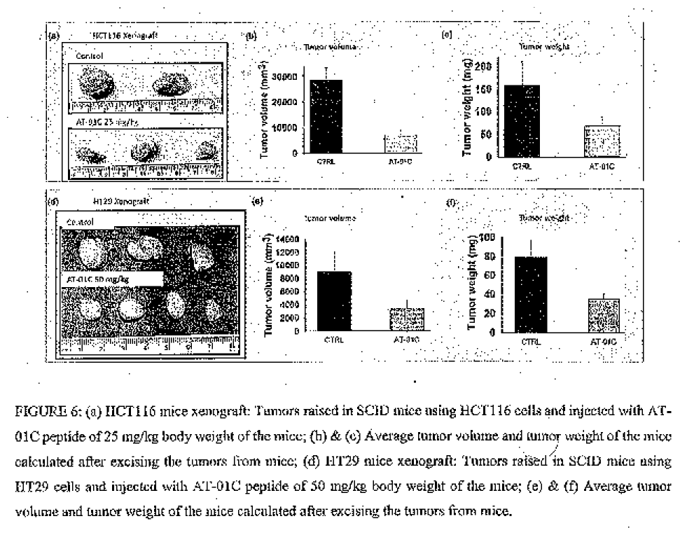

Tumors raised in SCID mice by subcutaneously injecting either 1 million HCT116 cells or HT29 cells (Xenograft model). After tumors developed and were visible to eyes AT-01C peptide treatment was started. For tumors raised using HCT116 colon cancer cells AT-01C of 25 mg/kg body weight of mice was injected intra-peritoneally. AT-01C peptide of 50 mg/kg body weight was injected intra-peritoneally as treatment for tumors raised with HT29 cells. AT-01C was injected daily surrounding the tumor 5 mm away from it. After 21 days of peptide injection, mice were sacrificed and tumors excised. The weight and volume of the tumors (both HCT116 & HT29 xenograft) were compared with their control counterparts where peptide was not injected. AT-01C injected mice xenografts showed decreased tumor volume and tumor weight. All the mice survived during the course of 21 days of the treatment (FIG. 5).

In conclusion, the invention provides a novel anti-cancer peptide AT01 or its genetic modification, derived from a secretary protein (MPT63) of M. tuberculosis. The mechanism of these peptides by stabilizing the expression of tumor suppressor protein SMAR1 is through prevention of ubiquitin mediated degradation. Stabilized expression of SMAR1 in turn confers the anti-cancer properties to these peptides such as anti-metastatic or anti-proliferation of cells. The potential of AT-01C to regress tumor volume and sizes indicates that it can be used as therapeutics for cancers. These novel peptides are therefore a promising therapeutics to cancers where SMAR1 is found to be modulated.

### Results

### Peptide Stabilizes SMAR1

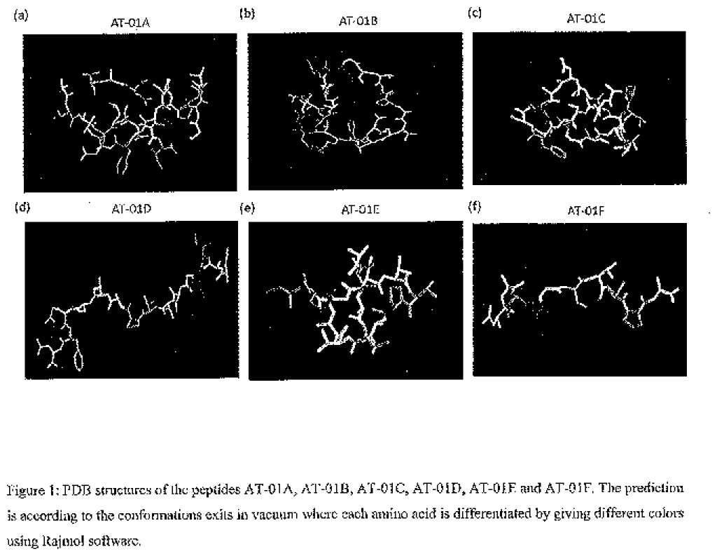

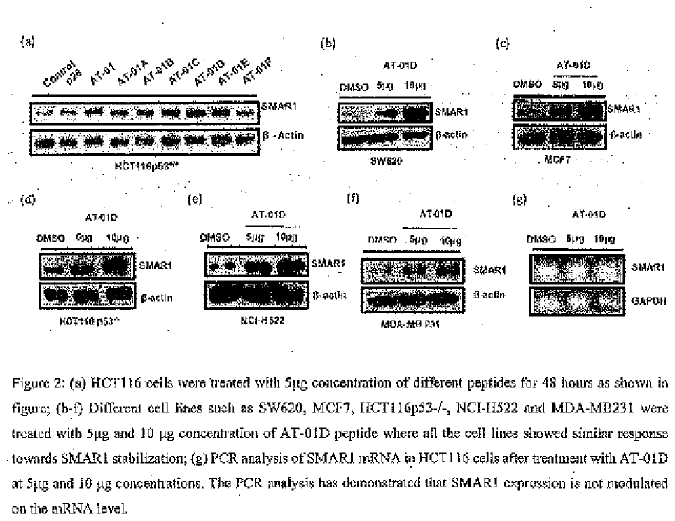

Since the pI of the peptides ranges from 6-10, hence, such peptides will behave differently according to varying pH of the environments or culture medium conditions (Table 1). Theoretically the molecular weight of each peptide was calculated using Peptide Calculator software. Predicted structures of the peptides are presented in FIG. 1. HCT116 cells treated with different available peptides for 48 hours stabilized SMAR1 expression. Some of the peptides failed to induce SMAR1 (FIG. 2a). We also tested another novel peptide p28, from Pseudomonas aeruginosa which has very well demonstrated the anticancer activity and is currently undergoing for Phase-I clinical trials. In-order to see the response of SMAR1 using these peptides, various cell lines of different cancer origin were treated with AT-01D as it stabilized SMAR1 the most. However, similar response was shown in different cell lines when tested with these peptides with different concentrations. Peptides were tested in MCF7, SW620, NCH-I522, HCT116p53^{-/-} and MDA-MB231 cells. Since induction of SMAR1 was observed in HCT116p53^{-/-} cells, it further proved that such peptide works independently of p53 (FIG. 2b-f). Further PCR analysis using AT-01D peptide failed to control the transcriptional regulation of SMAR1 (FIG. 2g). Hence, it is assumed that these peptides were able to stabilize the protein by preventing its degradation at protein level.

### Mechanism of SMAR1 Induction

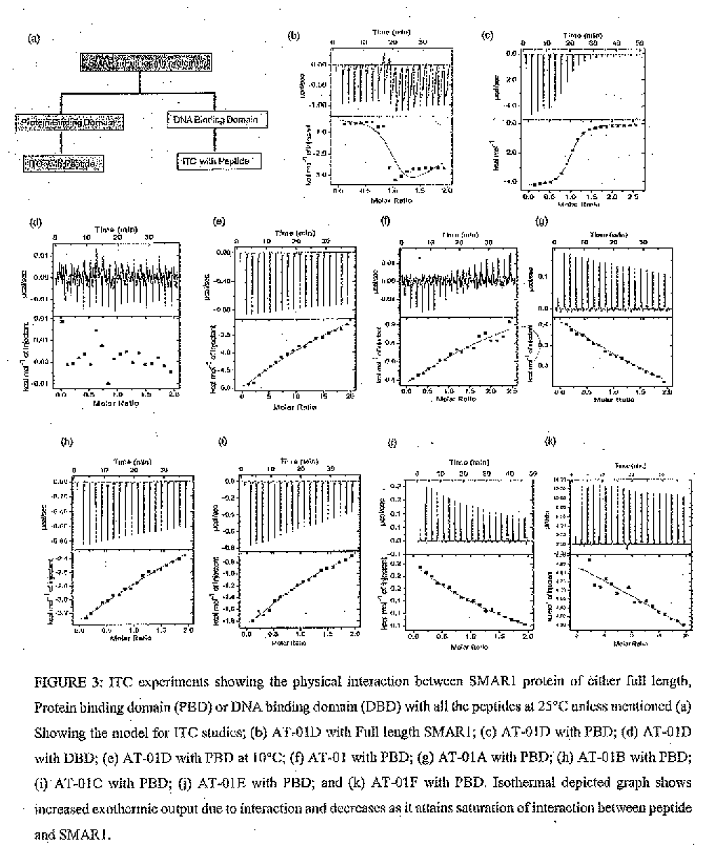

Since SMAR1 stability is reportedly not observed at transcription level, studies were focused on the possibility of peptide regulation with SMAR1 protein. To check any possible interaction of the peptides and SMAR1, Isothermal Titration calorimetry (ITC) was performed. Most of the peptides showed interaction with the Protein Binding Domain (PBD) of SMAR1, but failed to bind with DNA Binding Domain (DBD). Except AT-01, all the peptides showed either weak or strong interaction with PBD of SMAR1 as showed in the FIG. 3. One of the possible explanations is down-regulation of SMAR1 in higher grades of breast cancer as reported earlier by Rampalli et al (2005). At protein level down-regulation of protein occurs by proteosomal degradation mechanisms. It is possible that these peptides might bind and block the ubiquitin binding sites of SMAR1 thereby preventing degradations.

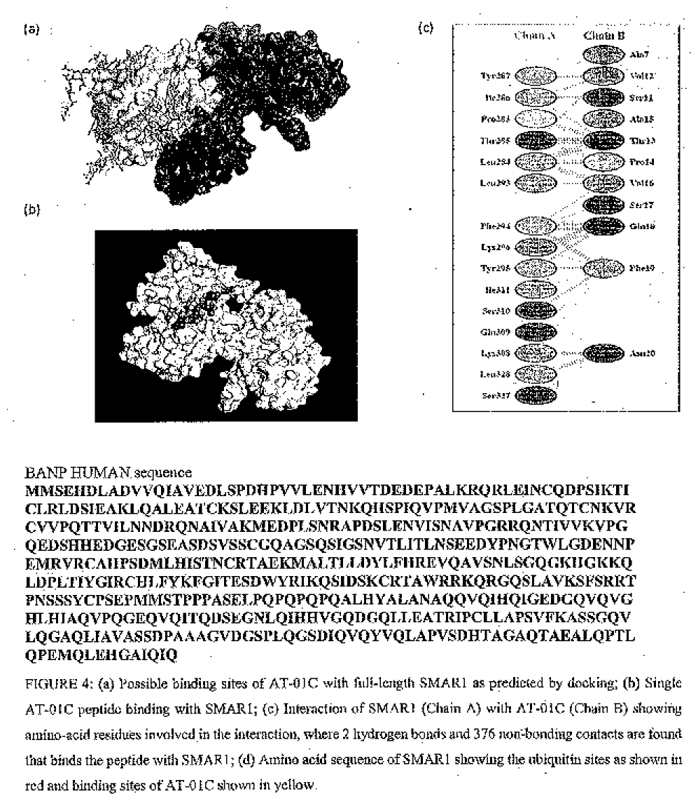

To support the ITC studies, peptides were docked with SMAR1 using bio-informatics software (FIG. 4). PDD structures of all the peptides were created using AntiCP software. Peptides were then subjected to docking with SMAR1 PDB structure. From the analysis, the interacting residues/amino acids were identified using PDB sum. Bond length and nature of the interaction was studied to confirm and validate the peptide and SMAR1 docking. Two hydrogen bonds and 376 non-bonding contacts were found. In the SMAR1, amino acid sequences RCHL and RQRL are the ubiquitin sites. AT-01C binds to L and blocks the site which is very much essential for ubiquitin moiety to bind and perform degradation of SMAR1. Unlike for ITC, full length PDB structure of SMAR1 was used for docking purpose. There is a good co-relation of the interaction results found in both ITC and docking studies. The protein binding potentials of all the peptides are almost the same as seen from Table 1. Hence, for binding to any proteins it will bind with similar binding potential. From such studies it is found that interaction of the peptides and SMAR1 depends on the steric hindrance and conformation of the structures.

### Peptides Delayed Cell Migration and Colony Formation Assay

To check the anti-cancer (anti-metastatic) properties of these peptides, wound healing or cell migration assay was performed. Treatment of highly metastatic MDA-MB231 breast cancer cells using AT-01D showed that this peptide is able to attenuate the metastasis or migration of these cells as compared to the control DMSO treated cells. MDA-MB231 cells were treated with AT-01C and AT-01D for cell migration assay. It is observed that both these peptides could attenuate migration of MDA-MB231 cells when recorded up to 16 hours (FIG. 5 a-c). These peptides appear to regulate genes responsible for cell migration. Analysis from the Annexin V staining by Flow cytometry (FACS) after treatment with AT-01C and AT-01D showed no signs of cell death. Hence, attenuation of cell migration is sole responsible for the anti-metastatic or cell arrest of SMAR1 and not due to apoptosis of the cells.

HCT116 cells were treated with AT-01D every day and subjected to Colony formation assay. After 12 days of culture, colonies formed were found to be more in the DMSO (Control) treated than in the AT-01D treated cells. AT-01D peptide delayed or decreased the rate of cell proliferation into a colony formation in due course of time as seen from the figure and graph (FIG. 5, d-f). No apoptotic cells or group of cells observed till the 12^{th} day of culture in the DMSO or AT-01D treated cells. 50 cells in a group were considered to be one colony. Malonia et al (2011) had earlier reported that SMAR1 controls cell proliferation through cell cycle regulatory proteins by stabilizing tumor suppressor protein p53 and controlling the fate of oncogenes like Cyclin D1 down-regulation (Rampalli et al., 2005). These two factors contribute to slow proliferation of cells leading to delayed colony formation unit in due course of peptide treatment. In cancer, SMAR1 gets degraded and loss of cell cycle control regulation leads to uncontrolled cell proliferation. If SMAR1 degradation in cancers can be prevented by introduction of any small molecule compounds or peptides like AT-01C and AT-01D, cell proliferations can be controlled. This brings a promising therapeutics at least in those cancers where SMAR1 has been identified to be de-regulated.

The mechanisms and pathways responsible for SMAR1 down-regulation are still unclear. Loss of tumor suppressor protein SMAR1 results in more metastatic and aggressiveness of cancer cells. Earlier reports suggest that Prostaglandin A2 mediated down-regulation of Cyclin D1 is due to stabilized SMAR1 expression. Such compounds target 5′UTR of SMAR1 and cause mRNA stability (Malonia et al., 2011; Singh et al., 2007). SMAR1 also associates with other tumor suppressor proteins like p53 and mediates cell cycle arrest or tumor progressions. In absence of SMAR1 various transcription factors and tumors suppressor proteins may not function efficiently to prevent the tumorigenic properties of cancers.

It is found that M. tuberculosis derived secretory protein MPT63 modified peptides are able to stabilize the expression of SMAR1. From experiments it is clear that modification of the peptides from the parent peptide can change the stability of SMAR1. AT-01 failed to interact with SMAR1 as seen from the ITC and docking studies leading to very less or no induction of SMAR1. However, AT-01C and AT-01D could significantly induce expression of SMAR1, showing positive results obtained during ITC and docking studies. It is not clear the cause of SMAR1 stability upon peptide treatment but is assumed that peptides dock onto the ubiquitin sites of SMAR1 preventing protein degradation. From bioinformatics analysis, it is found that SMAR1 ubiquitin sites are blocked by the interacting peptides at RCHL. SMAR1 harbors three ubiquitin sites that allow ligases to bind and therefore degradation prevails. Even though the origin of the peptides is same, modification to amino acid length leads to different structural conformations. Loops formed by the peptides are seen to dock onto the SMAR1 protein. This maintains the SMAR1 stability partially and prevents degradation of the protein.

The anti-cancerous properties of the peptides can be attributed to stabilized expression of SMAR1. From the cell migration assay, it is confirmed that some of these M. tuberculosis derived peptides demonstrate the capacity to delay the metastatic properties of invasive cancer cell lines like MDA-MB231. Earlier SMAR1 has been reported to delay metastasis by down-regulating TGFβ pathway which is very active in cancers like breast, colon etc. (Singh et al., 2007). Although, the direct effect of these peptides on TGFβ pathway has not been checked, stabilized SMAR1 expression due to the peptides definitely throw light in the anti-metastatic activity. Colony formation assay supports the potential of these peptides to decrease cell proliferations.

In conclusion, the invention provides a novel anti-cancer peptide AT01 or its genetic modification e.g., AT-01C and AT-01D, derived from a secretary protein (MPT63) of M. tuberculosis. The mechanism of these peptides by stabilizing the expression of tumor suppressor protein SMAR1 is through prevention of ubiquitin mediated degradation. Stabilized expression of SMAR1 in turn confers the anti-cancer properties to these peptides such as anti-metastatic or anti-proliferation of cells. These novel peptides are therefore a promising therapeutics to cancers where master cell regulator SMAR1 is found to be stabilized.

### REFERENCES

1. Cancer Facts & FIGS. 2015, available online at http://www.cancer.org/acs/groups/content/@editorial/documents/document/acspc-044552.pdf (accessed Mar. 2, 2016).
2. Levine, A J. 1997. p53, the Cellular Gatekeeper for Growth and Division. Cell 88, 323-331.
3. Michael, D., and Oren, M. 2002. The p53 and Mdm2 families in cancer. Curr. Opin. Genet. Dev. 12, 53-59.
4. Vogelstein, B., Lane, D., and Levine, A J. 2000. Surfing the p53 network. Nature 408, 307-310.
5. Oren, M. 2003. Decision making by p53: life, death and cancer. Cell Death. Differ. 10, 431-442.
6. Giaccia, and Kastan. 1998. The complexity of p53 modulation:emerging patterns from divergent signals. 2973-2983.
7. Michael, D. and M. Oren. 2003. The p53-Mdm2 module and the ubiquitin system. Semin.

Cancer Biol. 13:49-58.
8. Egleton, R D., and Davis, T P. 1997. Bioavailability and Transport of Peptides and Peptide Drugs into the Brain. Peptides 18, 1431-1439.
9. Goulding C W, Parseghian A, Sawaya M R, Cascio D, Apostol M I, Gennaro M L, Eisenberg D. 2002. Crystal structure of a major secreted protein of Mycobacterium tuberculosis-MPT63 at 1.5-A resolution. Protein Sci. 12:2887-2893.
10. Malonia S K, Sinha S., Lakshminarasimhan P., Singh K., Jalota-Badhwar A, Rampalli S., Kaul-Ghanekar R., Chattopadhyay S. 2011. Gene regulation by SMAR1: Role in cellular homeostasis and cancer. Biochimica et Biophysica Acta 1815, 1-12.
11. Jalota A., Singh K., Lakshminarasimhan P., Kaul-Ghanekar R., Jameel S. and Chattopadhyay S. 2005. Tumor Suppressor SMAR1 Activates and Stabilizes p53 through its Arginine-Serine-rich Motif. J. of Biol. Chem. 280, 16019-16029.
12. Rampalli S, Lakshminarasimhan P, Bhatt A, Kundu T K, Chattopadhyay S. 2005. Tumor suppressor SMAR1 mediates cyclin D1 repression by recruitment of the SIN3/histone deacetylase 1 complex. Mol. Cell Biol. 19:8415-8429.
13. Singh K., Mogare D., Obula R., Gogiraju G R, Pande G, Chattopadhyay S. 2007. p53 Target Gene SMAR1 Is Dysregulated in Breast Cancer: Its Role in Cancer Cell Migration and Invasion. Plos One. 2.660
14. J. R. Robinson, Sustained and Controlled Release Drug Delivery Systems, ed. (Marcel Dekker, Inc., New York, 1978).

1-10. (canceled)

11: A biologically active peptide having an amino acid residue sequence which is a member of the group consisting of
ATATVNAIRGSVTPAVSQFNARTAD;
GQVWEATATVNAIRGSVTPAVSQFN;
ATATVNAIRGSVTPAVSQFN;
NAIRGSVTPAVSQFN;
ATATVNAIRGSVTPA;
NAIRGSVTPA;
GNVIRGSVAPAVSQFNGPL;
PQNAIRGAVTPAVSQFNVA;
PQNAIRWSVTPAVSQFNLH;
DFNAIVGSVTPAVSQFNGAL;
LKNPIRGSQTPAVSQFNHIG;
GANAIRGSHDPAVSQFNGL ;
GANAIRCSVFPAVSQFNIG;
GYPVAGQVWEATATVNAIRGSVTPAVSQFNARTADGINYR;
and
PQGEQSTGKIYFDVTGPSPT.

12: A peptide suitable as anticancer agent and having an amino acid residue sequence which is a member of the group consisting of
ATATVNAIRGSVTPAVSQFNARTAD;
GQVWEATATVNAIRGSVTPAVSQFN;
ATATVNAIRGSVTPAVSQFN;
NAIRGSVTPAVSQFN;
ATATVNAIRGSVTPA;
and
NAIRGSVTPA.

13: The peptide in accordance with claim 12 having the amino acid residue sequence ATATVNAIRGSVTPAVSQFN.

14: The peptide in accordance with claim 12 having the amino acid residue sequence NAIRGSVTPAVSQFN.

15: The peptide in accordance with claim 12 having the amino acid residue sequence GQVWEATATVNAIRGSVTPAVSQFN.

16: The peptide in accordance with claim 12 having the amino acid residue sequence ATATVNAIRGSVTPA.

17: A pharmaceutical composition comprising a peptide of claim 11 and a pharmaceutically acceptable carrier therefor.

18: The pharmaceutical composition of claim 17 which is a solution containing said peptide in a concentration in the range of about 0.1 to about 250 milligrams/milliliter.

19: The pharmaceutical composition of claim 18 further containing a buffer and having a pH value in the range of about 5 to about 7.

20: A method for stabilizing Scaffold Matrix Attachment Region Binding Protein 1 (SMAR1) which comprises administering to a patient in need thereof a stabilizing amount of a peptide of claim 11.

21: The method according to claim 20 wherein the peptide has the amino acid residue sequence ATATVNAIRGSVTPAVSQFN.

22: The method according to claim 20 wherein the peptide has the amino acid residue sequence NAIRGSVTPAVSQFN.

23: The method according to claim 20 wherein the peptide has the amino acid residue sequence GQVWEATATVNAIRGSVTPAVSQFN.

24: The method according to claim 20 wherein the peptide has the amino acid residue sequence ATATVNAIRGSVTPA.

25: A peptide in accordance with claim 11 for use as an anticancer agent.

26: A peptide in accordance with claim 11 for use as stabilizing agent for Scaffold Matrix Attachment Region Binding Protein 1 (SMAR1).

27: A pharmaceutical composition according to claim 17 for the treatment of cancer.

28: A pharmaceutical composition according to claim 18 for the treatment of cancer.

29: A pharmaceutical composition according to claim 19 for the treatment of cancer.

30: A pharmaceutical composition according to claim 17 for stabilizing Scaffold Matrix Attachment Region Binding Protein 1 (SMAR1).
